(19) United States
(12) Patent Application Publication
(10) Pub. No.: US 2005/0038474 A1
(43) Pub. Date: Feb. 17, 2005

(54) Implantable automatic defibrillator with subcutaneous electrodes

(76) Inventor: Thomas J. Wool, Montgomery, AL (US)

Correspondence Address: OLSON & HIERL, LTD., 36th Floor, 20 North Wacker Drive, Chicago, IL, 60606, US

(21) Appl. No.: 10/137,185

(22) Filed: Apr. 30, 2002

(51) Int. Cl. A61N 1/39

(52) U.S. Cl. 607/5

(57) ABSTRACT

An implantable automatic cardioverter defibrillator system having a subcutaneous housing, one lead and subcutaneous electrode, the housing comprising the other electrode. Alternatively, the system has a subcutaneous housing, two leads and two respective subcutaneous electrodes.

### FIELD OF THE INVENTION

The present invention relates generally to implantable cardiac stimulators, and more particularly to an implantable automatic defibrillator.

### BACKGROUND INFORMATION

An implantable automatic cardioverter defibrillator (IACD) can be implanted in a patient who has been identified as being likely to suffer cardiac arrhythmias, such as ventricular tachycardia or ventricular fibrillation which can cause sudden death. The IACD detects the occurrence of ventricular fibrillation or other cardiac arryhthmia and automatically delivers appropriate therapy. IACD's in their most general form include appropriate electrical leads and electrodes for collecting electrical signals generated by the heart, and for delivering electric pulses or shocks to the heart to provide cardioversion or defibrillation therapy. Also included are batteries, energy storage capacitors, and control circuitry for sensing the electrical activity of the heart, for charging the capacitors and for triggering the delivery of therapeutic electrical pulses or shocks through the leads and electrodes. IACD's can also include circuitry for providing pacing therapy for treating bradycardia.

Defibrillation therapy generally involves rapid delivery of a relatively large amount of electrical energy to the heart at high voltage. Presently available batteries suitable for use in IACD's are not capable of delivering energy at such levels directly. Consequently, it is customary to provide a high-voltage energy storage capacitor that is charged from the battery via appropriate charging circuitry. To avoid wasting battery energy, the high-voltage energy storage capacitor is not maintained in a state of charge, but rather is charged during an interval after fibrillation has been identified by the control circuitry, and immediately prior to delivering the shock.

Early concepts of implantable defibrillators, such as disclosed in Reissue U.S. Pat. No. 27,652 by Mirowski et al., envisioned an electrode system employing a ventricular endocardial electrode and an epicardial electrode mounted to the heart or a plate electrode implanted subcutaneously. Implantation of an epicardial electrode requires a thoracotomy.

It would be desirable to produce an implantable defibrillation system which entirely avoids the necessity of a thoracotomy, and the development of such systems is disclosed in U.S. Pat. No. 4,727,877 issued to Kallok; U.S. Pat. No. 4,708,145 issued to Tacker et al.; and U.S. Pat. No. 5,099,838 issued to Bardy.

Other endocardial defibrillation electrodes are disclosed in U.S. Pat. No. 4,481,953 issued to Gold et al.; U.S. Pat. No. 4,161,952 issued to Kinney et al.; U.S. Pat. No. 4,934,049 issued to Kiekhafer et al; U.S. Pat. No. 4,641,656 issued to Smits; and U.S. Pat. No. 5,042,143 issued to Holleman et al. The Kinney, Gold, Kiekhafer and Holleman et al. patents all disclose endocardial defibrillation leads employing defibrillation electrodes fabricated from elongated coils of biocompatible metal, mounted exposed to the exterior of the defibrillation lead, for location in the right ventricle and other locations within the heart. The Smits and the Bardy patents both disclose a variety of endocardial defibrillation electrodes intended for use in the atrium, ventricle and coronary sinus, all of which employ electrodes taking the form of elongated coils of conductive biocompatible metals.

The endocardial leads set forth in the above cited references are generally employed with one or more additional endocardial or subcutaneous electrodes. In general, there has been a trend toward lead systems employing three or more such electrodes in order to reduce defibrillation thresholds to an acceptable level. In the Tacker and Kallok references, lead systems which employ three or more electrodes sequentially paired with one another are discussed. In the Bardy and the Smits patents, lead systems in which three or more electrodes are used simultaneously to deliver a defibrillation pulse are disclosed.

The subcutaneous leads employed in the systems as discussed above may be fabricated using metal mesh electrodes, as disclosed in U.S. Pat. No. 4,765,341, issued to Mower et al., coiled metal wire electrodes as disclosed in U.S. Pat. No. 4,817,634, issued to Holleman et al. or may be the metal enclosure of the defibrillator as disclosed in the above-cited Kallok patent.

A variety of pulse wave forms and polarities have been suggested. Monophasic capacitive discharge pulses are disclosed in the above cited Mirowski reissue patent. Biphasic pulses are disclosed in U.S. Pat. No. 4,953,551, issued to Mehra et al. Damped sinusoidal pulses are disclosed in U.S. Pat. No. 4,834,100, issued to Charms.

A return to lead systems employing only two electrodes is suggested in U.S. Pat. No. 4,922,927, issued to Fine et al. This patent proposes the use of an electrode system as in the above-cited Mirowski reissue Patent, using a right ventricular electrode and a subcutaneous electrode, which may correspond to prior art subcutaneous electrodes or may be the metal enclosure of the defibrillator. The right ventricular electrode carries an elongated coil electrode fabricated of a copper-zirconium alloy coated with iridium oxide. The use of biphasic pulses in such a two electrode system is also recommended. The Fine patent states that defibrillation thresholds as low as 7-10 joules may be achieved with such an endocardial lead in conjunction with a subcutaneous electrode, apparently implanted in proximity to the ventricles rather than pectorally.

Other available technology includes external cardiac pacemaker-defibrillators that work through a pair of external, transcutaneous patch electrodes placed on the skin on the front and back of the chest such that electrical current can flow through the heart during use. Alternatively, both patch electrodes can be placed anteriorly. Such external devices are employed for emergency resuscitation or with hospitalized patients who have already had a cardiac event. It would be impractical to use external electrodes for continuous monitoring and automatic defibrillation of an ambulatory patient as it could not be assured that the electrodes would be affixed and properly connected at all times.

Currently available IACD's are expensive and their use is generally restricted to individuals who have survived a cardiac arrest or have undergone electrophysiological studies that indicate that they are in a very high risk category for cardiac arrest. Unfortunately, this leaves a much larger population of individuals who are generally recognized as being at increased risk for sudden cardiac death or cardiac arrest who don't meet current criteria for these devices.

A study published in The New England Journal of Medicine, Vol. 346, No. 12, pp. 877-883, discusses the benefits of prophylactic implantation of a defibrillator in patients with myocardial infarction and reduced left ventricular ejection fraction. The findings show that the implantation of a defibrillator improves survival, and prophylactic implantation of a defibrillator is recommended in such patients.

An editorial in the same issue of the above cited journal, at pp. 831-833, refers to the expanding indications for implantable cardiac defibrillators being demonstrated by ongoing studies, but notes that the cost effectiveness of defibrillator prophylaxis remains in question and looms as a barrier to the wider us of that approach. The editorial mentions the hope of investigators that the manufacture of lower-cost defibrillators made especially for prophylactic use will make the approach more cost effective.

If a device that were easy to implant and relatively inexpensive were available, it would have a much greater applicability than the currently available versions. A basic device would be effective at providing defibrillation and backup pacing without all of the advanced features of the more expensive transvenous devices that are currently available. A basic model could be implanted in any patient who was thought to be at risk for sudden cardiac death without having to meet the current stringent requirements. If such a patient later was determined to require more advanced therapy in the future, then one of the more expensive, sophisticated transvenous devices could then be implanted.

It would be desirable to provide an implantable automatic cardioverter defibrillator that is easily implanted and that avoids the trauma of a thoracotomy and that also avoids the sometimes difficult placement of transvenous leads. Such desirable advantages, and others, are provided by the present invention.

### SUMMARY OF THE INVENTION

In one aspect, the present invention includes an automatic defibrillation system having an implantable automatic defibrillator. A pair of subcutaneous patch electrodes, suitable for being implanted subcutaneously, are each connected to a respective one of a pair of electrical leads that are operably connectable to the defibrillator.

In another aspect, the present invention includes an implantable automatic defibrillation system having an implantable automatic defibrillator with a housing having a subcutaneous electrode. A subcutaneous patch electrode, suitable for being implanted subcutaneously, is connected to an electrical lead that is operably connectable to the defibrillator.

According to other aspects of the invention, an automatic defibrillation system is implanted with the defibrillating electrodes placed subcutaneously outside the rib cage.

Other aspects of the invention will be apparent from the following description of preferred embodiments made with reference to the drawings.

### BRIEF DESCRIPTION OF THE DRAWINGS

FIG. 1 is a prior art implantable automatic cardioverter defibrillator shown implanted with epicardial electrodes in a patient.

FIG. 2 is an embodiment of the present invention shown implanted with subcutaneous patch electrodes in a patient.

FIG. 3 is a cross-sectional view of a patient in whom the embodiment of FIG. 2 is implanted.

FIG. 4 is another embodiment of the present invention shown implanted with one subcutaneous patch electrode and the housing comprising the other electrode.

FIG. 5 is a cross-sectional view of a patient in whom the embodiment of FIG. 4 is implanted.

FIG. 6 is a cross-sectional view of a subcutaneous patch electrode useful in connection with the present invention.

### DETAILED DESCRIPTION OF THE PREFERRED EMBODIMENTS

The present invention in one preferred embodiment involves an implantable automatic cardioverter defibrillator (“IACD”) or a basic defibrillation-only device having leads connected to subcutaneous patch electrodes that can be placed in subcutaneous pockets over the front and back of the chest, with the IACD implanted, for instance, in an abdominal subcutaneous pocket. In another preferred embodiment, the housing of the IACD itself comprises one of the electrodes and is implanted pectorally.

A device according to the present invention typically would not be used in a patient who would require frequent or continuous pacing or cardioversion, or frequent defibrillation. Nor would it typically be used in a patient who had a high likelihood of requiring pacing, cardioversion or defibrillation in the very near future. A more typical candidate for implantation of a device according to the present invention would be a member of a larger population who are at some risk for sudden cardiac death but who do not meet current criteria for transvenous or intrathoracic devices. The medical literature suggests that the number of individuals who actually die from sudden cardiac arrest or arrhythmia is many times greater than the number who meet the criteria for receiving currently available devices.

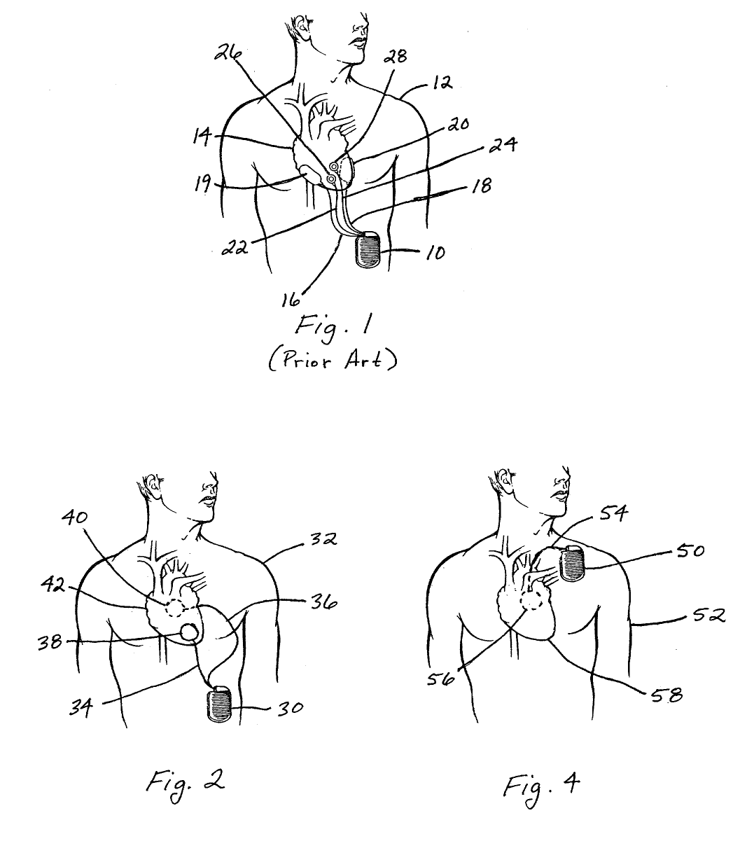

Referring to FIG. 1, a prior art implantable automatic cardioverter defibrillator (“IACD”) 10 is shown implanted subcutaneously in the abdominal region of a patient 12. A number of leads having epicardial terminal electrodes extend from the hermetically sealed housing of IACD 10 and are affixed to the heart 14. Leads 16 and 18 terminate in epicardial patch electrodes 19 and 20 that are affixed to the anterior and posterior surfaces, respectively, of the ventricles of heart 14. Cardioverting or defibrillating electrical pulses or shocks are delivered by IACD 10 through leads 16 and 18 and electrodes 19 and 20 to convert tachycardia or fibrillation to a normal rhythm. Leads 22 and 24 terminate in epicardial sensing electrodes 26 and 28 that are affixed to the anterior surface of the ventricles of heart 14. Sensing electrodes 26 and 28 sense electrical signals naturally generated by the heart during normal pumping contractions. The sensed signals are conveyed through leads 22 and 24 to IACD 10, where control circuitry analyzes the signals and determines whether therapeutic pulses or shocks are needed. Because the electrodes 19, 20, 26 and 28 of the prior art device of FIG. 1 are implanted epicardially in contact with the heart 14, a thoracotomy is necessary to gain surgical access to the heart so that the leads can be affixed.

The present invention eliminates the need for a thoracotomy and also eliminates the need for the tedious and sometimes risky procedure of implanting transvenous leads.

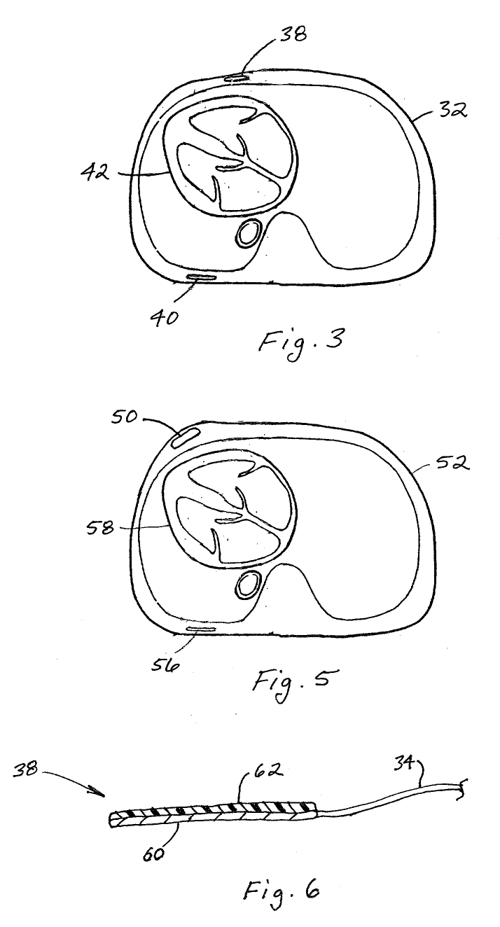

Referring to FIGS. 2 and 3, a first preferred embodiment of the present invention is illustrated. An implantable automatic cardioverter defibrillator (“IACD”), or basic defibrillation-only device, 30 is implanted subcutaneously in the abdominal region of a patient 32. IACD 30 can include backup pacing capability, if desired. A pair of leads 34 and 36 extend from the hermetically sealed housing of IACD 30 and terminate in respective subcutaneous patch electrodes 38 and 40. Subcutaneous electrode 38 is implanted anteriorly of the heart 42 in a subcutaneous pocket outside the rib cage of the patient 32. Subcutaneous electrode 40 is implanted posteriorly of the heart 42 in a subcutaneous pocket that is likewise outside the rib cage. Consequently, it is not necessary to enter the chest via a thoracotomy to implant the device of FIGS. 2 and 3. Leads 34 and 36 are placed subcutaneously between the IACD and the patch electrodes by conventional subcutaneous tunneling techniques using a catheter and/or trocar.

Referring to FIGS. 4 and 5, a second preferred embodiment of the present invention is illustrated. An implantable automatic cardioverter defibrillator (“IACD”), or basic defibrillation-only device, 50 is implanted subcutaneously in the pectoral region of a patient 52 outside the rib cage. IACD 50 can include backup pacing capability, if desired. A single lead 54 extends from the hermetically sealed housing of IACD 50 and terminates in a subcutaneous patch electrode 56. Subcutaneous electrode 56 is implanted posteriorly of the heart 58 in a subcutaneous pocket outside the rib cage of the patient 52. The housing of IACD 50 itself comprises one electrode of the system with electrode 56 comprising the other. The housing of IACD 50 can be made of conductive metal such as titanium or surgical stainless steel, as is customary, or alternatively a patch electrode can be secured to the outside of the housing of IACD in case the housing is constructed of a non-conductive material.

As with the first embodiment discussed above, it is not necessary to enter the chest via a thoracotomy to implant the device of FIGS. 4 and 5. Lead 54 is placed subcutaneously between the IACD and the patch electrode by conventional subcutaneous tunneling techniques using a catheter and/or trocar.

Referring to FIG. 6, patch electrode 38 and a portion of corresponding lead 34 are shown in cross-section. The other patch electrodes 40 and 56 and respective leads 36 and 54, discussed above, are similarly constructed. Patch electrode 38 has an electrically conductive, preferably biocompatible metal, layer 60 electrically connected to lead 34. Overlying conductive layer 60 is an electrically insulating layer 62, preferably biocompatible plastic material such as polyurethane. Patch electrode 38 is implanted subcutaneously with the conductive layer 60 facing the rib cage, and the insulating layer 62 facing the skin. This construction and arrangement minimizes the effect of the electrical shock on overlying tissue.

In use, either embodiment of the IACD or basic defibrillation-only device can be surgically implanted through a cutaneous incision into a subcutaneous pocket. Likewise, a patch electrode can be surgically implanted through a cutaneous incision into a subcutaneous pocket. A second patch electrode can be so implanted if desired. A catheter and/or trocar can be used to tunnel subcutaneously between the pocket for the IACD or basic defibrillation-only device and the pocket for the subcutaneous patch electrode. The lead can be placed subcutaneously through the tunnel and mechanically and electrically connected at each end to the patch electrode and to the defibrillator. Preferably, the lead as manufactured is already electrically connected and hermetically sealed to the patch electrode. In that case, the tunneling takes place from the subcutaneous pocket for the patch electrode toward the subcutaneous pocket for the defibrillator. The free end of the lead is then extended through the tunnel and mechanically and electrically connected to the defibrillator using conventional standard connectors.

While the present invention has been described in terms of preferred specific embodiments, no limitation on the invention is thereby intended. The scope of the invention is set forth in the appended claims.

1. An implantable automatic defibrillation system comprising:
an implantable automatic defibrillator;
a pair of electrical leads operably connectable to the defibrillator; and
a pair of subcutaneous patch electrodes suitable for being implanted subcutaneously, each operably connected to a respective one of the pair of electrical leads.

2. The defibrillation system of claim 1, wherein the patch electrodes have a conductive layer and an insulating layer.

3. An implantable automatic defibrillation system comprising:
an implantable automatic defibrillator having a housing comprising a subcutaneous electrode;
an electrical lead operably connectable to the defibrillator; and
a subcutaneous patch electrode suitable for being implanted subcutaneously and operably connected to the electrical lead.

4. The defibrillation system of claim 3, wherein the patch electrode has a conductive layer and an insulating layer.

5. An implantable automatic defibrillation system comprising:
implantable means for automatic defibrillation;
a plurality of electrical leads operably connectable to the implantable means for automatic defibrillation; and
a plurality of subcutaneous patch electrode means suitable for being implanted subcutaneously for conducting an electrical impulse to tissue; and
electrical lead means operably connected to the patch electrode means and operably connectable to the implantable means for automatic defibrillation for conducting an electrical pulse from the implantable means for automatic defibrillation to each patch electrode means.

6. The defibrillation system of claim 5, wherein the patch electrode means have a conductive layer and an insulating layer.

7. An implantable automatic defibrillation system comprising:
implantable means for automatic defibrillation having a housing comprising a subcutaneous electrode;
patch electrode means suitable for being implanted subcutaneously for conducting an electrical impulse to tissue; and
electrical lead means operably connected to the patch electrode means and operably connectable to the means for automatic defibrillation for conducting an electrical pulse from the implantable means for automatic defibrillation to the patch electrode means.

8. The defibrillation system of claim 7, wherein the patch electrode means has a conductive layer and an insulating layer.

9. A method of implanting an automatic defibrillation system comprising:
providing an implantable automatic defibrillator, a pair of electrical leads operably connectable to the defibrillator, and a pair of subcutaneous patch electrodes suitable for being implanted subcutaneously, each patch electrode operably connected to a respective one of the pair of electrical leads;
implanting the defibrillator subcutaneously;
implanting the subcutaneous patch electrodes subcutaneously;
implanting the leads subcutaneously; and
operably connecting the leads to the defibrillator.

10. The method of claim 9, wherein one electrode is implanted anterior of the heart and the other electrode is implanted posterior of the heart.

11. The method of claim 9, wherein both electrodes are implanted anterior of the heart.

12. The method of claim 9, wherein the leads are implanted by subcutaneous tunneling.

13. The method of claim 9, wherein the patch electrodes are implanted outside the rib cage.

14. The method of claim 9, wherein each patch electrode is operably connected to a terminal end of a respective electrical lead.

15. A method of implanting an automatic defibrillation system comprising:
providing an implantable automatic defibrillator having a housing comprising a subcutaneous electrode, an electrical lead operably connectable to the defibrillator, a subcutaneous patch electrode suitable for being implanted subcutaneously and operably connected to the electrical lead;
implanting the defibrillator subcutaneously;
implanting the subcutaneous patch electrode subcutaneously;
implanting the lead subcutaneously; and
operably connecting the lead to the defibrillator.

16. The method of claim 15, wherein the defibrillator is implanted pectorally anterior of the heart.

17. The method of claim 15, wherein the patch electrode is implanted posterior of the heart.

18. The method of claim 15, wherein both the defibrillator and the patch electrode are implanted anterior of the heart.

19. The method of claim 15, wherein the lead is implanted by subcutaneous tunneling.

20. The method of claim 15, wherein the patch electrode is implanted outside the rib cage.

21. The method of claim 15, wherein the patch electrode is operably connected to a terminal end of a respective electrical lead.

22. The method of claim 15, wherein the implantable automatic defibrillator is capable of delivering backup pacing pulses through the patch electrode.
